(19) United States
(12) Patent Application Publication
(10) Pub. No.: US 2009/0240148 A1
(43) Pub. Date: Sep. 24, 2009

(54) ULTRASONIC APPARATUS AND METHOD FOR REAL-TIME SIMULTANEOUS THERAPY AND DIAGNOSIS

(75) Inventors: Jong Seob Jeong, Los Angeles, CA (US); Jin Ho Chang, Torrance, CA (US); K. Kirk Shung, Monterey Park, CA (US)

Correspondence Address: MCDERMOTT WILL & EMERY LLP, 2049 CENTURY PARK EAST, 38th Floor, LOS ANGELES, CA, 90067-3208, US

(73) Assignee: UNIVERSITY OF SOUTHERN CALIFORNIA, Los Angeles, CA (US)

(21) Appl. No.: 12/407,580

(22) Filed: Mar. 19, 2009

Related U.S. Application Data
(60) Provisional application No. 61/038,002, filed on Mar. 19, 2008.

(51) Int. Cl. A61B 8/13; A61N 7/00

(52) U.S. Cl. 600/439

(57) ABSTRACT

For noninvasive treatment of tissue using high intensity focused ultrasound, composite imaging and therapy acoustic techniques are described. Embodiments include an integrated multi-functional confocal array and a strategy to perform both imaging and therapy simultaneously with this array by using coded excitation techniques with/without a notch filter. An exemplary array embodiment includes a triple-row phased array with one array in the center row for imaging and two arrays in the outer rows for therapy. Different types of piezoelectric materials and stack configurations may be employed to maximize the respective therapy and imaging functionalities. Reflected therapeutic signals that would otherwise corrupt the quality of imaging signals received by the center-row array can be mitigated or removed by use of the coding and/or a notch filter when B-mode images are formed during therapy. A 13-bit Barker code is preferred for implementing coded excitation, although other codes or compression techniques may be used.

### RELATED APPLICATION

This application claims the benefit of U.S. Provisional Patent Application No. 61/038,002 entitled “Ultrasonic Apparatus and Method for Real-Time Simultaneous Therapy and Diagnosis,” filed 19 Mar. 2008, the entire contents of which are incorporated herein by reference.

### BACKGROUND

In recent years, high intensity focused ultrasound (“HIFU”) has become increasingly important in the noninvasive treatment of malignant tissues. Several clinical studies have been conducted to investigate the feasibility of HIFU treatment for breast, liver, and prostate cancer. HIFU therapy is usually performed in cooperation with medical imaging modalities such as magnetic resonance imaging (“MRI”), ultrasound imaging, and computed tomography (“CT”) in order to select and monitor a treatment region. MRI provides a high-resolution image and an efficacious temperature map, but it is expensive and requires a large space. Ultrasound is another common tool for image guidance. It offers advantages in real-time imaging, cost-effectiveness, excellent portability, and potential integration with other devices.

FIG. 1 depicts an acoustic stack of transducer 100A for diagnosis and an acoustic transducer 100B for treatment. Generally, for some other ultrasound techniques ultrasonic an imaging transducer consists of a matching layer, a piezoelectric layer, and a backing layer for achieving wider bandwidth. A matching layer with a quarter-wavelength thickness reduces impedance difference between a transducer and a tissue. Most piezoelectric layers, which change mechanical energy to electrical energy and vice versa, are made of a piezoelectric ceramic, e.g., lead zirconate titanate (“PZT”). A backing layer usually made from mixing epoxy with a metal powder attenuates the ultrasound wave transmitted into the backing layer. In case of transducer for treatment, its configuration is different from diagnostic transducer. Because matching layer might be affected by high temperature of piezoelectric material and the backing layer might decrease the intensity of ultrasound, usually only piezoelectric material is used to make a HIFU transducer.

Since treatment time is relatively long taking 2˜4 hours, therapeutic region might be misaligned easily due to patient's movement or breathing. The most effective solution to this problem is to carry out therapy and imaging the treatment region at the same time. For this purpose, the ultrasound imaging guided HIFU (“US-guided HIFU” or “US HIFU”) is preferred due to its capability of real-time imaging with a reasonable resolution, cost-effectiveness, excellent portability, and potential integration with another modality. There have been many attempts to develop real-time simultaneous UI-guided HIFU systems with limited success.

HIFU focuses high intensity ultrasound beam on the area to be treated using either thermal or mechanical effect resulting from considerable energy deposition at focal area. To achieve highly precise noninvasive surgery using HIFU, simultaneous targeting and monitoring functions are required for US-guided HIFU. There have been several attempts to develop a real-time simultaneous US-guided HIFU system. Several investigators have proposed a system equipped with two spatially separated transducers for treatment and imaging. Yet another paper reported integration of therapeutic and diagnostic functions into a single transducer array based on dual-mode operation, switching between treatment and diagnosis. However, these techniques have achieved limited success in real-time therapeutic and imaging capability. Spatially separated transducers may miss a target due to misalignment between two transducers. Implementation of switching mode using a single transducer array may degrade the performance of both treatment and diagnosis because the piezoelectric material and configuration of HIFU transducers are generally different from these of diagnostic transducers.

What is desired, therefore, are improved ultrasonic techniques for both diagnosis (imaging) and treatment of tissues.

### SUMMARY

The present disclosure in general terms is directed to novel apparatus and methods utilizing an integrated transducer design for true real-time simultaneous imaging and HIFU while maintaining treatment capability. The integrated acoustic transducer may be composed of multifunctional linear arrays, in which the center array row may be used for imaging and the outer row arrays may be used for therapy. Therapy can be performed with either continuous wave (“CW”) or coded signal like chirps. In addition, coded signals can be used for real-time imaging to minimize interference that arises from fundamental or harmonics of reflected therapeutic signal when the therapy and imaging are performed at the same time.

An aspect of the present disclosure includes system and methods using coded excitation with/without a notch filter for imaging purposes. Exemplary embodiments can utilize Barker codes for such coding/compression, however, not only the Barker code but also other codes techniques such as, but not limited to, chirp, and Golay code, can be used as an imaging signal. Exemplary embodiments can include with use of a notch filter for discriminating the reflected imaging energy from the therapeutic signals. The techniques can be applied to several targets such as breast, liver, prostate, and so on.

A further aspect of the present disclosure is directed to novel acoustic transducers that include an imaging array and one or more therapy arrays. Not only several types of arrays such as phased, linear, convex, and concave but also single element transducer can be used for this configuration. The arrays can be integrated together and fabricated so as to share a common focal point. An exemplary embodiment includes an integrated multi-functional confocal phased array (“IMCPA”) having an imaging phased array and two or more therapy phased arrays.

One skilled in the art will appreciate that embodiments and/or portions of embodiments of the present disclosure can be implemented in/with computer-readable storage media (e.g., hardware, software, firmware, or any combinations of such), and can be distributed over one or more networks. Steps or operations (or portions of such) as described herein, including processing functions to derive, learn, or calculate formula and/or mathematical models utilized and/or produced by the embodiments of the present disclosure, can be processed by one or more suitable processors, e.g., central processing units (“CPUs”), digital signal processors (“DSPs”), programmable logic devices (“PLDs”), and field programmable gate arrays (“FPGAs”) implementing suitable code/instructions in any suitable language (machine dependent on machine independent).

While aspects of the present disclosure are described herein in connection with certain embodiments, it is noted that variations can be made by one with skill in the applicable arts within the spirit of the present disclosure and the scope of the appended claims.

### BRIEF DESCRIPTION OF THE DRAWINGS

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIG. 1 depicts types of separate imaging and therapy acoustic transducers;

FIG. 2 depicts a diagrammatic view of an ultrasound system for simultaneous ultrasound imaging and therapy, in accordance with exemplary embodiments of the present disclosure;

FIG. 3 depicts a diagrammatic view of simultaneous imaging and therapy acoustic signal flow, in accordance with exemplary embodiments of the present disclosure;

FIG. 4 depicts a diagrammatic view of a collection of simulations for a therapeutic signal and a received signal for imaging in accordance with an embodiment of the present disclosure;

FIG. 5 depicts a diagrammatic view of a collection of simulations for the output of pulse compression signal processing, in accordance with an embodiment of the present disclosure;

FIG. 6 depicts a diagrammatic representation of a method according to an exemplary embodiment of the present disclosure;

FIG. 7 depicts a simplified schematic diagram of a front view (A) and side view (B), respectively of an IMCPA transducer with an imaging array and two therapy arrays, in accordance with exemplary embodiments of the present disclosure;

FIG. 8 depicts a set of graphs of imaging signals, as obtained with an IMCPA of the present disclosure, with CW interference signals;

FIG. 9 depicts signal processing for real-time imaging during therapy by using an IMCPA in accordance with exemplary embodiments of the present disclosure;

FIG. 10 depicts a graph of frequency responses of 4 MHz and 8 MHz notch filters, utilized for exemplary embodiments of the present disclosure;

FIG. 11. depicts a set of point target simulations with imaging signals superimposed with CW interference signals, in accordance with an exemplary embodiment of the present disclosure;

FIG. 12 depicts a set of simulated point target images of interference-mixed imaging signals after notch filtering, in accordance with an embodiment of the present disclosure.

FIG. 13 depicts a diagrammatic view of an experimental setup system used to collect echo data of (A) imaging signals and (B) CW signals as interference signals, in accordance with exemplary embodiments of the present disclosure;

FIG. 14 depicts a set of graphs (A-F) showing experimental frequency responses of imaging signals with CW interference signals, in accordance with an exemplary embodiment of the present disclosure; and

FIG. 15 depicts a set of graphs (A-C) showing experimental envelope signals, in accordance with an embodiment of the present disclosure.

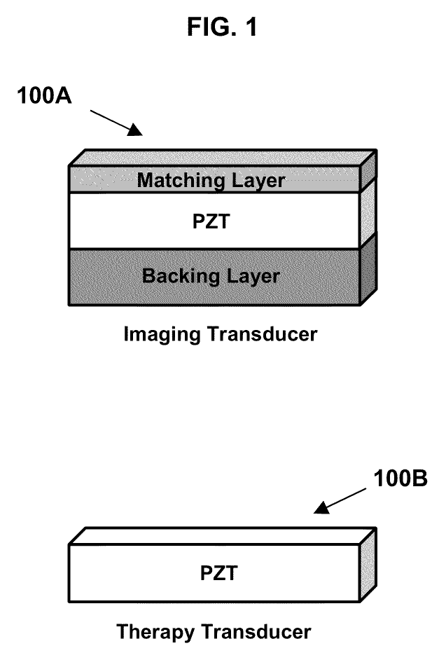

While certain embodiments depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

In general terms, the present disclosure is directed to novel apparatus and methods utilizing an integrated transducer design for true real-time simultaneous imaging and HIFU while maintaining treatment capability. The integrated transducer may be composed of multifunctional linear arrays, e.g., in which the center array row may be used for imaging and the outer row arrays may be used for therapy. Therapy can be performed with either continuous wave or coded signal, e.g., frequency-modulated “chirps.” In addition, several coded signals can be used for real-time imaging to minimize interference that arises from fundamental or harmonics of reflected therapeutic signal when the therapy and imaging are performed at the same time. Suitable coded excitation or pulse compression techniques such as various phase-modulated codes (e.g., Barker code) or frequency-modulation techniques (e.g., chirps) can be used for imaging purposes during therapy.

Embodiments of the present disclosure can provide real-time imaging during therapy using not only a pulse wave (“PW”) but also a CW. For this purpose, exemplary embodiments of the present disclosure include a HIFU transducer called integrated multi-functional confocal phased array (“IMCPA”). The transducer consists of triple-row phased arrays, e.g., a 6 MHz array in the center row for imaging and two 4 MHz arrays in the outer rows for therapy. Specifications such as dimension, frequency, and focal depth of an exemplary transducer are described for an application to the treatment of prostate tissue since one of the most common applications of commercial US-guided HIFU systems is currently the treatment of prostate tissue.

A key issue addressed by embodiments of the present disclosure is the suppression of reflected therapeutic signals received by the center-row array that is used for imaging. In the absence of such, when PW or CW signals for treatment and pulsed signals for imaging are transmitted to a target at the same time, the imaging signal would likely be undetectable due to the high amplitude of reflected therapeutic signals. One simple way to solve this problem may be either to decrease the intensity of transmitted therapeutic signals or to increase the intensity of transmitted imaging signals. However, these are not practical solutions because the intensity of therapeutic signal should be large enough to produce thermal necrosis, and the intensity of diagnostic ultrasound must be below that mandated by the U.S. Food and Drug Administration (“FDA”). As a practical solution to these limitations, embodiments of the present disclosure include a coded excitation technique, with or without a notch filter to form a B-mode image during therapy. Through simulation studies and experimental results, it was demonstrated that such techniques can be used to effectively suppress the interference signals during brightness-mode (“B-mode”) imaging while therapy was being carried out.

To achieve real-time US-guided HIFU in embodiments of the present disclosure, two different types of acoustic transducers, imaging and therapy, are integrated appropriately and effectively. To achieve true real-time simultaneous therapy and imaging, both a dedicated transducer configuration and a proper signal processing scheme are provided. Thus, embodiments of the present disclosure overcome the challenge of how two different transducers are combined while respectively maintaining therapeutic and monitoring capability. Exemplary embodiments can be used for many applications, including therapy of tumor or benign disease in liver, breast, prostate, and uterus; hemostasis of internal bleeds and thrombolysis; and, enhanced drug delivery, to name a few. Embodiments of the present disclosure can consequently be used to mitigate or minimize the deterioration of image quality, which can arise from interference by harmonics such those developed from reflected therapy energy, by firing coded signals (e.g., code sequences like biphase Barker code) for imaging.

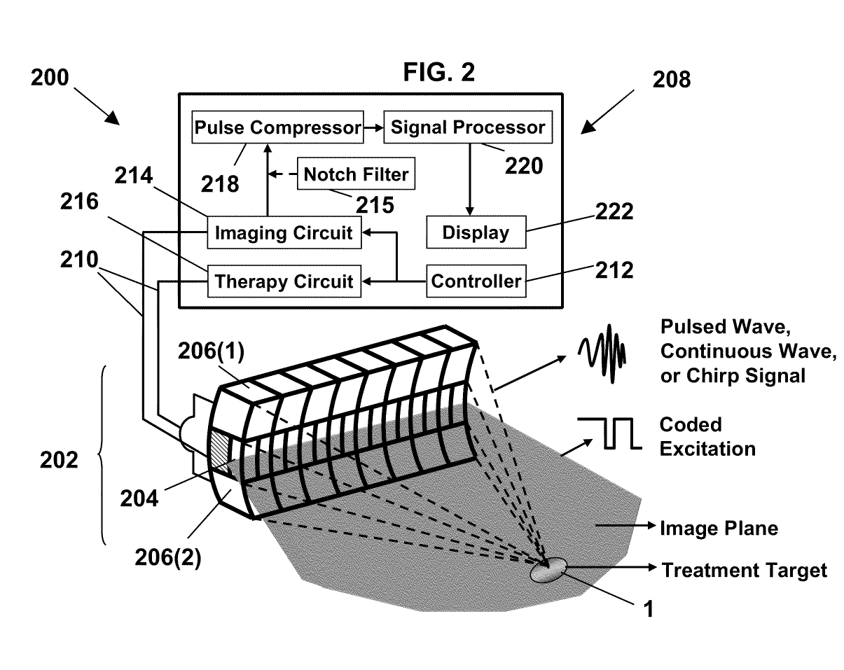

FIG. 2 depicts a diagrammatic view of an ultrasound system 200 for simultaneous ultrasound imaging and therapy of a targeted area 1, in accordance with exemplary embodiments of the present disclosure. FIG. 2 shows a transducer configuration 202 with coded excitation signal processing. The system 200 includes a compound (or dual-use) transducer array 202. The transducer array 202 includes an imaging array 204 and multiple therapy arrays 206(1)-206(2). The transducer arrays 204, 206(1)-206(2) can include individual transducers having suitable piezoelectric materials, e.g., lead ziconate titanate or the like. A controller system 208 can be connected to the array 202 by suitable connections 210.

The controller system 208 has circuitry/logic for controlling the acoustic output of the imaging array 204 and therapy arrays 206(1)-206(2). Controller system 208 can also include signal processing circuitry/logic for pulse compression (or coding) of the output of the imaging array 204 and for processing received acoustic echoes. For example, controller system 208 can include a controller 212, a therapy circuit 216, an imaging circuit 214, pulse compressor (hardware, firmware, and/or software) 218, and a signal processor 220 as shown. A display 222 can be included for displaying an image of the acoustic reflections after processing as ultrasound image. An optional notch filter 215 (hardware, firmware, and/or software) can be present for exemplary embodiments.

It will be appreciated that although FIG. 2 depicts a transducer array structure 202 combining only three-row arrays, in which the center row is indicated for use for imaging and the outer rows for therapy, the number of outer row arrays for the purpose of therapy can be increased depending on applications. Also, it can be possible to use one or more additional imaging arrays.

In operation of system 200, the two therapy arrays can emit signals (e.g., chirp signals) for treatment with a desired maximum intensity. The center frequency of therapeutic signal can also be selected/determined depending on applications. The center row (e.g., linear) array can transmit and receive coded signals for imaging during treatment.

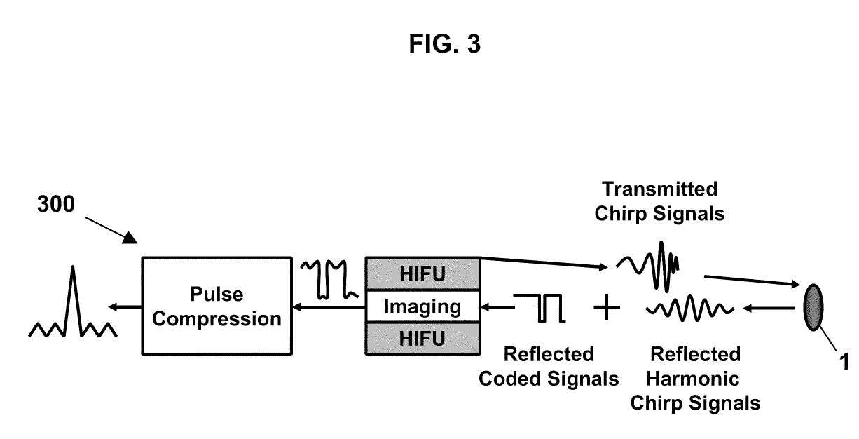

FIG. 3 depicts a diagrammatic view of simultaneous imaging and therapy acoustic signal flow 300, in accordance with exemplary embodiments of the present disclosure. As shown, an imaging array can transmit coded signals as imaging pulses to a target 1. Two therapy arrays (HIFU) on either side of the imaging array can transmit chirp signals to the target 1 for therapy. The imaging array receives from the target reflected coded signals plus reflected harmonic chirp signals. Due to the pulse compression, the deleterious effect of the reflected therapy harmonics on the coded signals can be mitigated.

The harmonics of reflected chirp signals, which degrade image quality due to the fact that they interfere with echoes for imaging, can be effectively suppressed by pulse compression, for example, as indicated in FIGS. 2-3. For example, when the center-row arrays for imaging, e.g., array 204 in FIG. 2, emit 13-length (or 13-bit) Barker code, which is one of the possible coded sequences, at 4 MHz center frequency and the outer-row arrays emit chirp signals at 2 MHz center frequency, the center-row arrays will receive 4 MHz second harmonic chirp signals within the imaging bandwidth. Because the correlation value between the Barker code and the second harmonic chirp signal is very low, the negative effect of the second harmonic chirp signal on image quality can be effectively removed after pulse compression for imaging. The case of using continuous waves for therapy has been shown by the present inventors to produce similar results.

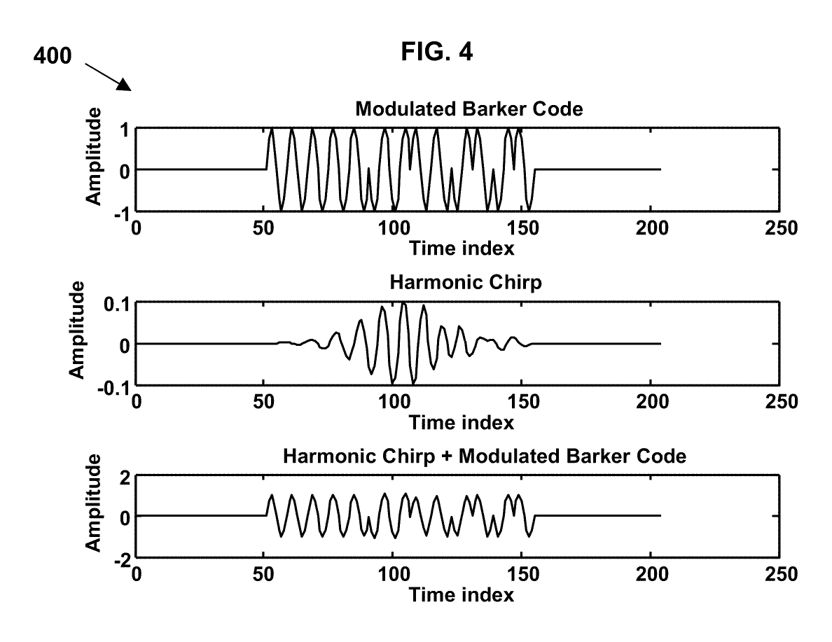

FIG. 4 depicts a diagrammatic view of a collection 400 of simulations for a therapeutic signal and a received signal for imaging in accordance with an embodiment of the present disclosure. FIG. 4 depicts a modulated Barker code (a), a reflected harmonic chirp (b), and a composite signal (c) with the modulated Barker code as affected by the received harmonic chirp.

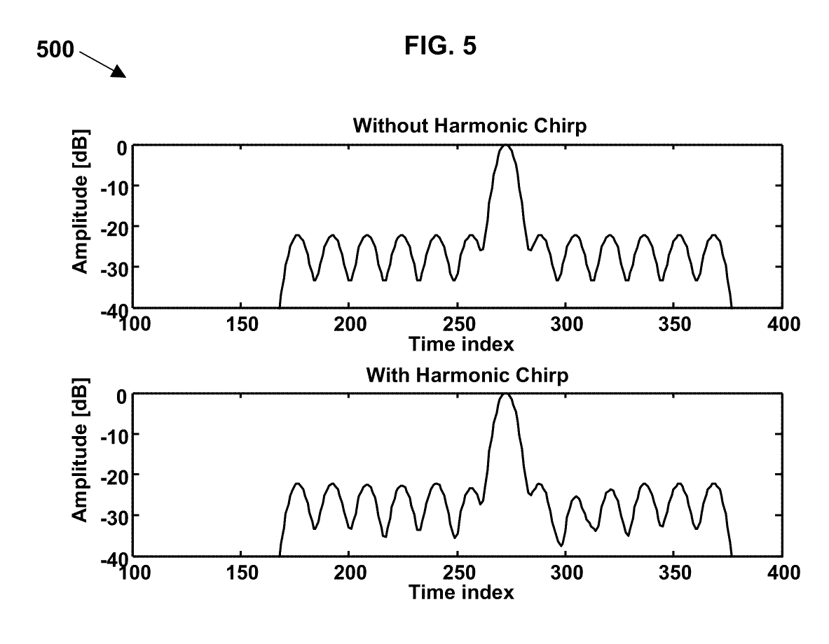

FIG. 5 depicts a diagrammatic view of a collection 500 of simulations for the output of pulse compression signal processing, in accordance with an embodiment of the present disclosure. The result from the pulse compression with Barker code contaminated by the harmonic chirp signals is a little bit distorted compared to pure standard Barker code, but it affects little the image quality because the range mainlobe width and sidelobe level are similar as shown in FIG. 5.

Exemplary embodiments of the present disclosure can utilize a sidelobe suppression filter with desired finite impulse response (“FIR”) filter tap values for improved sidelobe suppression. Because a sidelobe suppression filter, which can be used for pulse compression to increase signal to noise ratio (“SNR”) and contrast ratio, can decrease the sidelobe level of coded signal less than −40 dB, the amplitude of mixed signal with Barker code and harmonic chirp can be lower than −40 dB. This sidelobe level is seen as being reasonable considering typical sidelobe levels of diagnostic imaging for exemplary embodiments. Also, a mismatched filter to suppress sidelobe level can be used for other embodiments.

In accordance with the preceding descriptions, embodiments of the present disclosure can be used in situations when the amplitude of harmonics of therapeutic signals is below a relative range/limit (e.g., at least 10 dB lower than echo signal for imaging), reasonable SNR for high resolution diagnostic imaging. Because the therapeutic arrays do not coincide to the image plane (for exemplary embodiments) the amplitude of the harmonic chirp signals can be relatively small. Representative simulation results are shown in FIG. 4 and FIG. 5. Although the amplitude of the reflected therapeutic signal is higher than this range, a notch filter can be used as a technique to overcome such a limitation, as further described below.

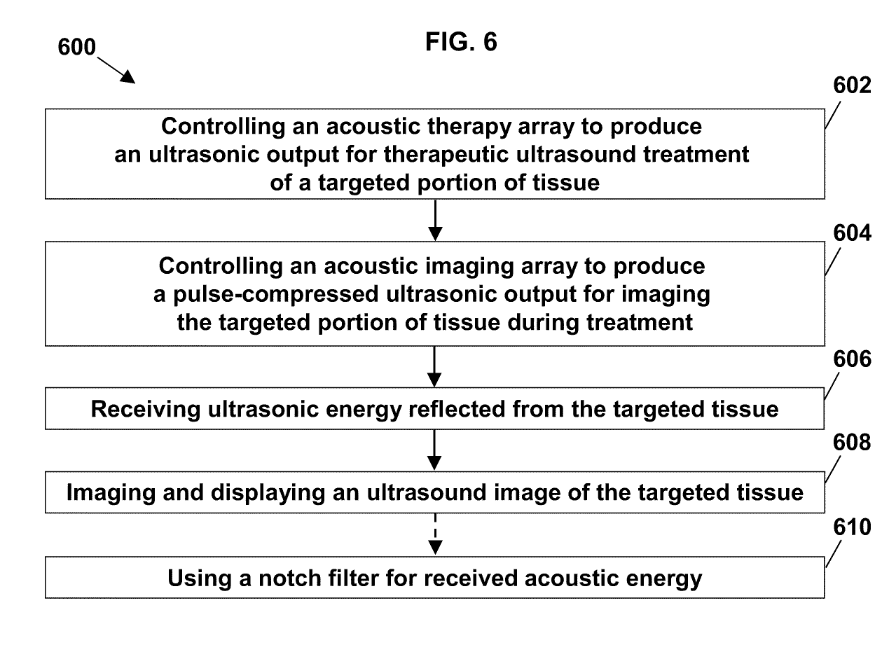

FIG. 6 depicts a box diagram of a method 600 of simultaneous ultrasonic imaging and therapy, in accordance with exemplary embodiments of the present disclosure. An acoustic therapy array can be controlled to produce an ultrasonic output for therapeutic treatment of a targeted portion of tissue, as described at 602. An acoustic imaging array can be controlled to produce a pulse-compressed ultrasonic output for imaging the targeted portion of tissue during treatment, as described at 604.

Continuing with the description of method 600, ultrasonic energy that is reflected from the targeted tissue can be received, as described at 606. The ultrasonic reflection signals can be processed and displayed (e.g., on a suitable display means or display) as one or more ultrasound images of the targeted tissue, as described at 608. A notched filter can optionally be used, as described at 610, and as further described below.

### EXAMPLE 1

### Integrate Multi-Functional Confocal Phased Array (“IMCPA”) Embodiment

For some embodiments described previously, there was an assumption for operation that the amplitude of the received therapy signal, namely, its harmonics, should be less than a relative threshold compare to that of the imaging signal, e.g., −10 dB than that of the imaging signal. Such an assumption may be reasonable for a diagnostic system. In the case of a HIFU system, such an assumption may not always be valid because the amplitude of the therapy signal is often too high resulting in high amplitude of interference signals. To overcome such a limitation, exemplary embodiments of the present disclosure can utilize a notch filter, regardless of amplitude of therapy signals.

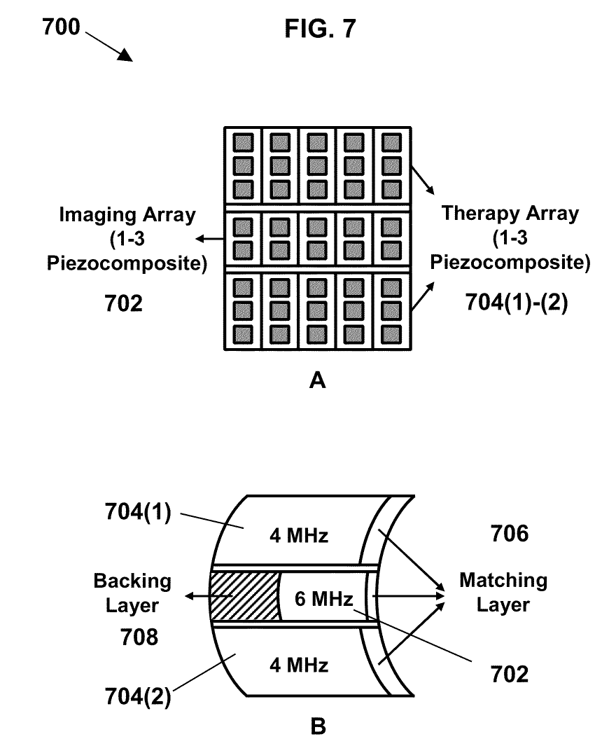

An exemplary embodiment of the present disclosure was fabricated as an integrated multi-functional confocal phased array (“IMCPA”) operational as a multi-row array transducer. FIG. 7 depicts a simplified schematic diagram of a front view (A) and side view (B), respectively of an IMCPA transducer 700 with an imaging array 702 of 5×2 elements and two therapy arrays 704(1)-704(2) of 5×3 elements. In FIG. 7(A), the front view of IMCPA 700 omits the matching layer. In FIG. 7B however, the side view of IMCPA 700 shows a matching layer. All arrays of 700 were fabricated to have 1-3 piezocomposite structures and their surfaces were constructed to have a common focal point in elevational direction. The center imaging array 702 has a backing layer, as shown, to increase the bandwidth and the outer therapy arrays have air backings to maximize transmission of ultrasound. A matching layer would also increase the transmission efficiency of IMCPA 700. Usually a matching layer is used in imaging transducers to improve its sensitivity. In the fabrication of the HIFU transducer, the application of a matching may be problematic. The area of the transducer surface can be too large to maintain uniform thickness and a uniform bonding line. Heating on the surface of the transducer may cause detachment of the layer from the transducer during high voltage operation. Thus, the application of a matching layer must be carefully considered for HIFU transducer with a very large dimension. But in this example, because the surface area of the transducer is not large, a matching layer may be used to increase transmission efficiency.

With continued reference to FIG. 7, the transducer 700 was composed of triple-row phased arrays including a 6 MHz array in the center row for imaging and two 4 MHz arrays in the outer rows for therapy as shown in FIG. 7. For HIFU therapy, a frequency range from 1 MHz to 4 MHz was preferred to increase thermal treatment effect. The frequencies in the range from 3 MHz to 4 MHz have been widely used for treatment of prostate cancer given the depth of penetration. Considering the requirements on efficient thermal necrosis and the depth of penetration (e.g., 4 cm˜5 cm for prostate), 4 MHz and 6 MHz frequencies were respectively chosen for treatment and imaging for the IMCPA. Since the second harmonic component of 4 MHz therapeutic signal would be generated at approximately 8 MHz, the imaging transducer should have an effective bandwidth of between 4 MHz and 8 MHz. Therefore, a 6 MHz transducer with a −6 dB fractional bandwidth of 50% is required for imaging.

For the purpose of efficient therapy and imaging with the IMCPA, the 6 MHz imaging array 702 of FIG. 7 was designed to have 128 elements with 0.73λ=188 μm pitch, 25 μm kerf, and 8 mm height, while each 4 MHz therapy array had 128 elements with 0.5λ=188 μm pitch, 25 μm kerf, and 14 mm height. The total dimension of IMCPA 700 of FIG. 7 was 24 mm×36 mm. These dimensions should be acceptable as an endocavity transducer. The dimension of the therapy 5 array is 24 mm×28 mm, which can generate an intensity of about 2000 W/cm² at a focal spot. The therapy array with half wavelength spacing between elements should be able to protect normal tissues from the grating lobes of HIFU beam. The f-numbers of lateral and elevational directions were 1.7 and 1.1, respectively, at a focal depth of 40 mm for the exemplary embodiment shown. The novel aspect of IMCPA is that all arrays are focused on the same region by a geometrically curved surface in the elevational direction eliminating the need for orienting transducers. The surface can be formed to have a desired shape, e.g., cylindrical, spherical, elliptical, etc. Also, a press-focused array is more efficacious than a lens-focused array because of acoustic energy absorption by a lens itself. It should be appreciated that while certain dimensions are described, such are merely representative, and other may of course be used.

In exemplary IMCPA designs, e.g., in accordance with FIG. 7, all arrays are preferably fabricated with a piezoelectric 1-3 composite for surface conformation and for its low acoustic impedance. However, the materials constituting 1-3 piezocomposite used for therapy transducers are typically different from those for imaging transducers. The piezoelectric materials with a high Curie temperature and a low dielectric/mechanical loss such as PZT4 and PZT8 are more desirable for therapy transducers. PZT-5H with a high dielectric constant and electromechanical coupling has been widely used for imaging array transducers. A high thermal resistance epoxy with a high glass transition temperature may be used for a composite therapy transducer resulting in improved temperature durability. A volume fraction ratio between piezoelectric material and epoxy of IMCPA of 75% was selected, given the specifications, which should be enough to generate the required acoustic intensity. A matching layer would reduce the acoustic impedance mismatch between the transducer and the body resulting in a high transmission efficiency. It is also advisable to construct a HIFU transducer without a backing layer to allow maximal energy transmission in the forward direction and to alleviate fabrication difficulties.

A strategy for fabricating the IMCPA 700 of FIG. 7 was to assemble together individual arrays after they are designed and fabricated separately. A press-focused method may be used before or after combining these transducers. Another advantage of this configuration is that the amplitude of reflected therapeutic signals received by the imaging transducer might be reduced due to an elevational angle difference between the therapy and the imaging arrays, causing most reflected therapeutic signals be directed toward the therapy arrays rather than the imaging array.

### EXAMPLE 2

### Signal Processing for Prostate Treatment Embodiment

In exemplary embodiments of the present disclosure, an IMCPA transducer such as transducer 700 of FIG. 7 can be utilized as an ultrasound transducer and system for real-time simultaneous therapy and diagnosis for noninvasive surgery, e.g., for prostate tissue. For such therapy, the spatial-peak temporal-average intensity (Iₛₚₜₐ) at a focal point is preferably higher than 1000 W/cm² to accomplish thermal necrosis. The PW with a high duty cycle can potentially be used if it can satisfy the requirement. Although embodiments of the present disclosure can employ PW as well as CW signals for therapy, a 4 MHz CW signal with a 100% duty factor so as to yield an intensity of 2000 W/cm² was used for simulations and experiments for exemplary embodiments described herein. Other duty factors may of course be used. For such embodiments, a 6 MHz 1-cycle short pulse was chosen for imaging. Its duty factor was 0.042% under the condition of a typical 2.5 kHz pulse repetition frequency (“PRF”) so that Iₛₚₜₐ could be 18.8 mW/cm² as a diagnostic intensity in accordance with the FDA guidelines. With these parameters, the peak pressures of the CW signal and the 1-cycle short pulse at a focal point were computed and found to be 7.75 MPa and 1.16 MPa, respectively, from the formula in Eq. 1:

$$P = \sqrt{\frac{2 {ZI}_{spta}}{{t}_{df}}} , \qquad ( Eq . 1 )$$

where Z is the acoustic impedance of water (1.5 MRayl) and t_{df} represents the duty factor. Given the two computed peak pressure values, transmit ultrasound pressures for both therapy and imaging were adjusted in all simulations and experiments. It was assumed that the amplitude of the second harmonic signal at 8 MHz was −10 dB less than that at its fundamental frequency.

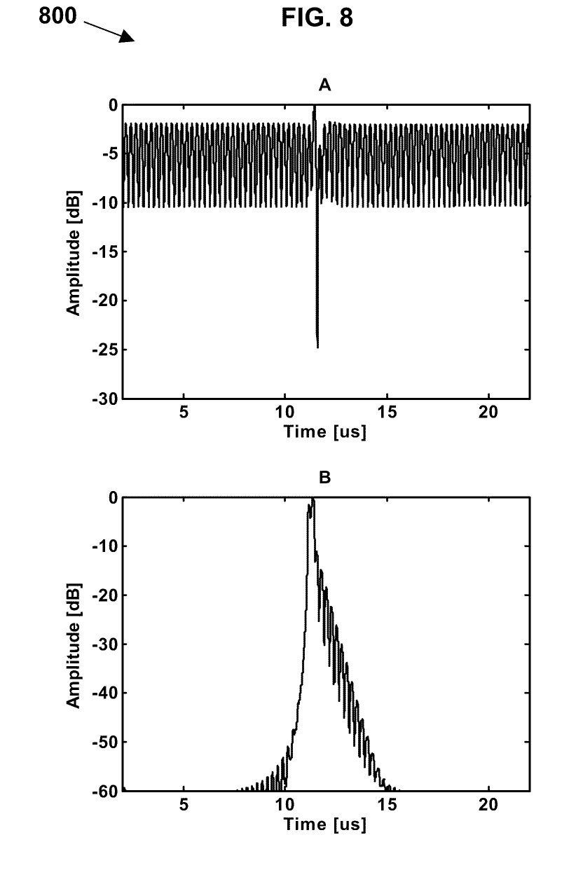

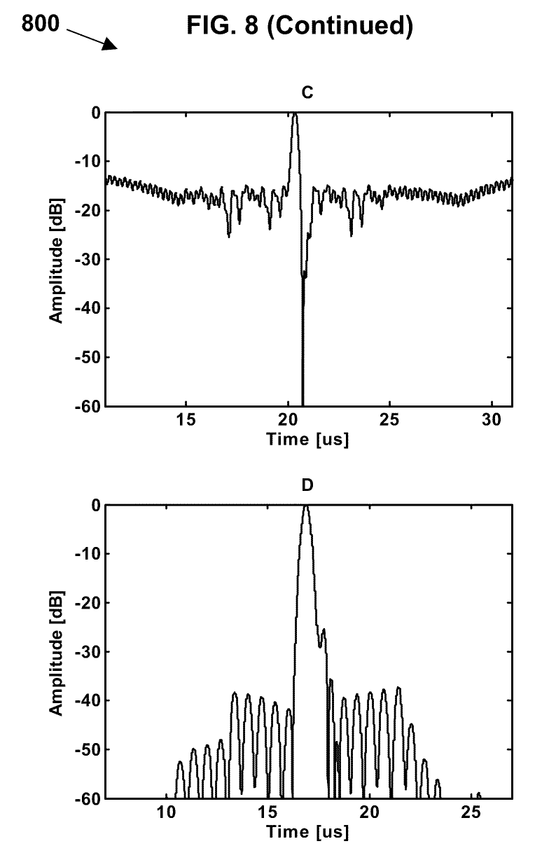

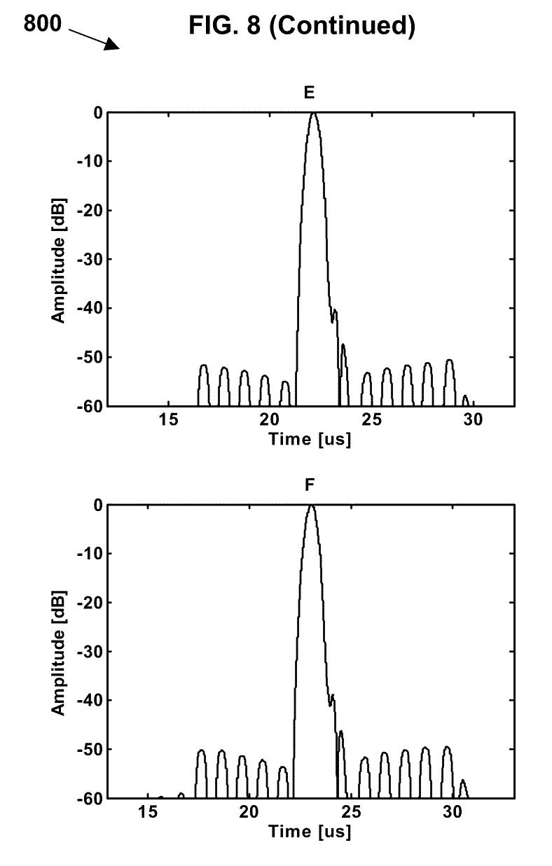

FIG. 8 depicts a set 800 of graphs of imaging signals, as obtained with an IMCPA of the present disclosure, with CW interference signals: 2-cycle short pulses, (A) before and (B) after notch filtering; the 13-bit Barker code with 2 cycles per bit, (C) before and (D) after notch filtering; and, the 13-bit Barker code with 3 cycles per bit, (E) before and (F) after notch filtering.

For such embodiments, it was found that when an IMCPA (e.g., array 700 of FIG. 7) fired 2-cycle short pulses for imaging and CW signals for therapy, the imaging array would receive echoes containing the high amplitude of 4 MHz and 8 MHz interference signals. This interference decreased the signal-to-noise ratio (“SNR”) of the imaging signals. The range sidelobe level of the envelope signal extracted from the echoes (FIG. 8A) was found to be approximately −2 dB, thus resulting in poor image quality. As a practical solution to this limitation, a coded excitation technique may be used for imaging with IMCPA designs because this technique may improve the SNR by increasing the average power without changing the peak power. (The easiest way to improve the SNR for imaging is to increase input power of the imaging array, but doing so could potentially violate the controlling FDA guidelines.) In addition, the correlation between coded imaging signals and CW interference signals is significantly lower than that when a short pulse signal is used for imaging.

Conventional coded or pulse-compressed excitation can employ frequency modulation schemes, e.g., chirps, and/or phase modulation schemes, e.g., Barker codes, and/or Golay codes to name a few examples. Among them, the Barker code is preferred for imaging in exemplary embodiments of the present disclosure due to its relatively simple hardware implementation and excellent robustness in noise suppression. The Barker code consists of N-bit or N-length biphase codes, and the optimal peak and range sidelobe level can be obtained from an autocorrelation function. Its range mainlobe width and sidelobe level depend on the number of bits and the number of sub-cycles per bit. By using a conventional sidelobe suppression filter, an acceptable sidelobe level, e.g., less than −40 dB for B-mode imaging, can be obtained. Currently, a 13-bit biphase code sequence (+1 +1 +1 +1 +1 −1 −1 +1 +1 −1 +1 −1 +1) is the largest length realized for the Barker code.

The mainlobe in the spectrum of the 13-bit Barker code with 1 cycle per bit goes beyond the frequency range from 4 MHz to 8 MHz. This broad frequency response results in a serious distortion of the mainlobe due to 4 MHz and 8 MHz reflected therapeutic signals. Since more than the 4-cycle per-bit Barker code might generate poor axial resolution, 2- and 3-cycle-per-bit Barker codes were considered for the experiment and simulation embodiments described herein. Other selections for the cycles per bit may be used.

As indicated in FIG. 8, when the 13-bit Barker code with 2 cycles per bit is used, the 4 MHz and 8 MHz CW interference signals can corrupt the received signal quality for imaging. The frequency distortion around 4 MHz and 8 MHz arising from the interference signals leads to a relatively high range sidelobe level, e.g., around −18 dB as shown in FIG. 8C. Although being 16 dB lower than the short pulse signal in FIG. 8A, this level may still not be enough to obtain an acceptable image quality. The 13-bit Barker code with 3 cycles per bit has about a −50 dB range sidelobe level in spite of the interference signals as shown in FIG. 8E. These two different results are due to the null point locations in their spectrums. The null points of the 3-cycle-per-bit Barker code are located around 4 MHz and 8 MHz, thus resulting in minimized mainlobe distortion. The −50 dB range sidelobe level of the 3-cycle-per-bit Barker code is acceptable for B-mode imaging. However, its axial resolution is poorer than that of the 2-cycle-per-bit Barker code. The −6 dB axial beamwidths of the 2- and 3-cycle-per-bit Barker code in FIGS. 8C and 8E are 0.27 mm and 0.49 mm, respectively. Therefore, the focus of the experiments and simulations described herein was on how the range sidelobe level of the 13-bit Barker code with 2 cycles per bit could be decreased to at least −40 dB in order to achieve a high axial resolution.

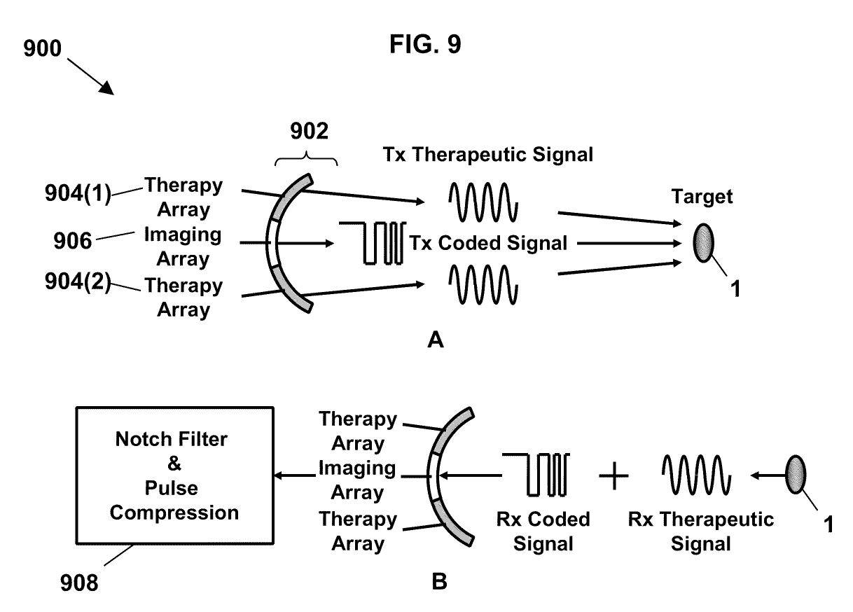

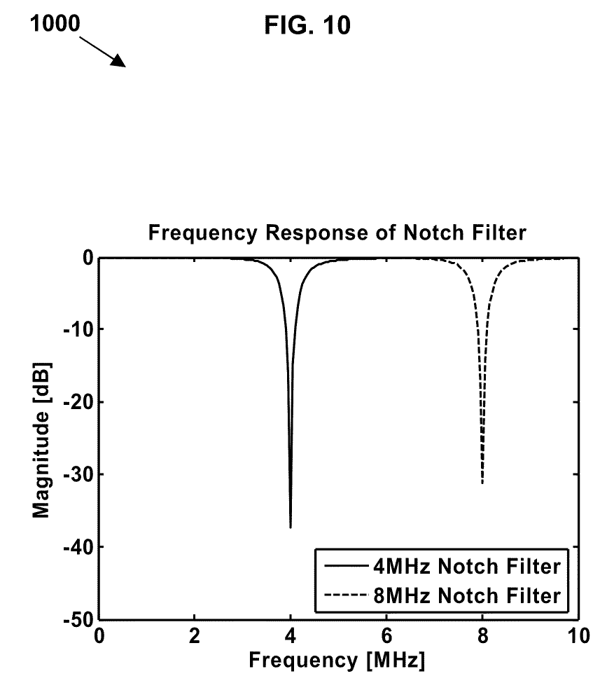

Fortunately, a reflected CW has a fixed frequency component, so that the known interference signal may be successfully minimized with a notch filter capable of rejecting a narrow band of frequency, as described in further detail below with respect to FIGS. 9-10.

FIG. 9 depicts a combined view 900 of signal processing for real-time imaging during therapy by using an IMCPA in accordance with exemplary embodiments of the present disclosure. FIG. 9 shows the transmission (A) and receptions (B) with an embodiment of a transducer array 902 of the present disclosure in which two outer therapy arrays 904(1)-904(2) transmit 4 MHz CW signals to a target 1. At the same time, an inner imaging array 906 is shown emitting 6 MHz coded signals similar to conventional sector scanning. The imaging array 906 receives the reflected coded signals along with reflected therapeutic signals. After pulse compression, the SNR may accordingly be improved, and is preferably less than −40 dB for B-mode imaging. In FIG. 9A, the therapeutic and coded imaging signals are emitted to the target at the same time. In FIG. 9B, the reflected therapeutic signal received by imaging array are shown removed by means of notch filtering and pulse compression 908.

A notch filter is widely used in radar or speech processing to attenuate CW signals at specific frequencies while nearby frequencies are relatively unaffected. A notch filter was designed using MATLAB (made commercially available by The MathWorks Inc., Natick, Mass.), for exemplary embodiments of the present disclosure, and notch attenuation values were found to be around −37 dB and −31 dB at 4 MHz and 8 MHz, respectively, as shown in FIG. 10.

FIG. 10 depicts a graph 1000 of frequency responses of 4 MHz and 8 MHz notch filters, utilized for exemplary embodiments of the present disclosure. The notch attenuation values are −37 dB and −31 dB at 4 MHz and 8 MHz, respectively.

With continued reference to FIG. 10, it can be noted that the sharpness of the notch filter depends on a quality factor, e.g., defined as the ratio of notch frequency over bandwidth of the notch filter. The quality factor should be properly determined by considering over shoot or under shoot in a pass band. In the experimental and simulation embodiments described herein, the quality factors for 4 MHz and 8 MHz were 7 and 14, respectively. The difference of 6 dB notch attenuation between two frequencies of the notch filter may be compensated by a 10 dB amplitude difference between fundamental and harmonic components of the interference signals.

This notch filter was applied to conventional 2-cycle short pulse signal, the 13-bit Barker code with 2 and 3 cycles per bit. The amplitude of interference signals was successfully suppressed after notch filtering in all cases. However, a serious frequency distortion of the short pulse around 4 MHz and 8 MHz generated undesired ripples in its envelope as shown in FIG. 8B. In the case of the Barker code with 2 cycles per bit, frequency distortions at around 4 MHz and 8 MHz were less than the short pulse signal since the locations of null points were close to 4 MHz and 8 MHz. The Barker code with 2 cycles per bit as in FIG. 8D had a −40 dB range sidelobe level which was 22 dB lower than the code without notch filtering, i.e., −18 dB shown in FIG. 8C. Since the locations of null points of the Barker code with 3 cycles per bit were close to interference frequencies, its range sidelobe level in FIG. 8F was similar to the pulse compression result without notch filtering (FIG. 8E). This may indicate that the null point plays a pivotal role in efficaciously decreasing the effect of reflected therapeutic signals on image quality. A notch filter can be utilized help to further decease the effect when the null points do not perfectly match the frequencies of the interference signals.

### EXAMPLE 3

### Simulation Results

A point target simulation was performed with the IMCPA design 700 of FIG. 7, using the Field II program, as made available by J. A. Jensen, “Field: A program for Simulating Ultrasound Systems,” Med. Biol. Eng, Comput., vol. 34, pp. 351-353, 1996, the entire contents of which are incorporated herein by reference. Other suitable software can be used for comparable simulations. The design parameters of IMCPA 700, described previously, were used in this simulation.

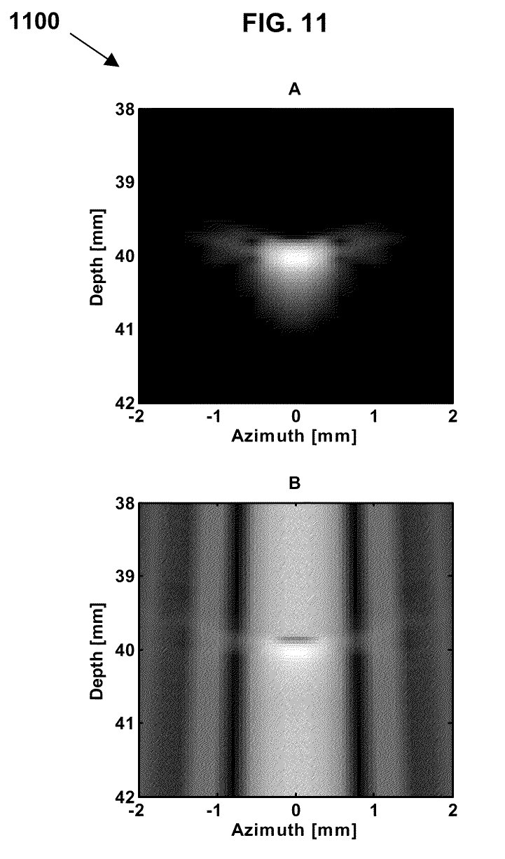

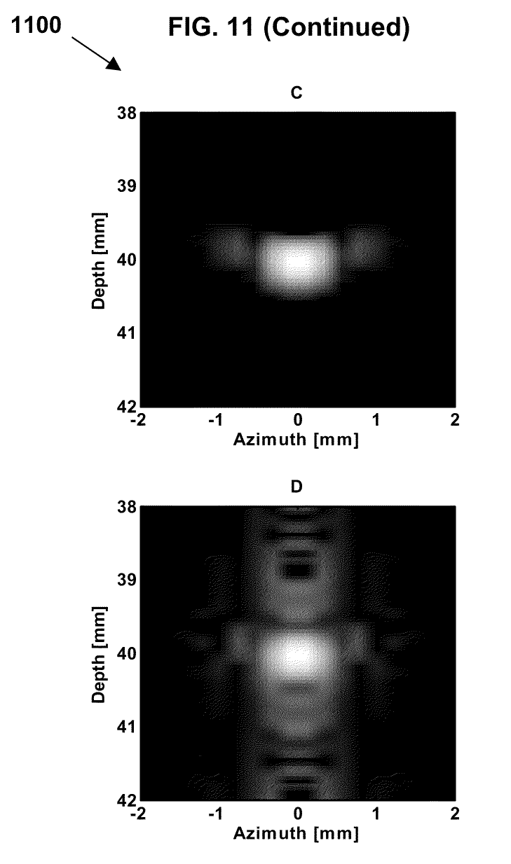

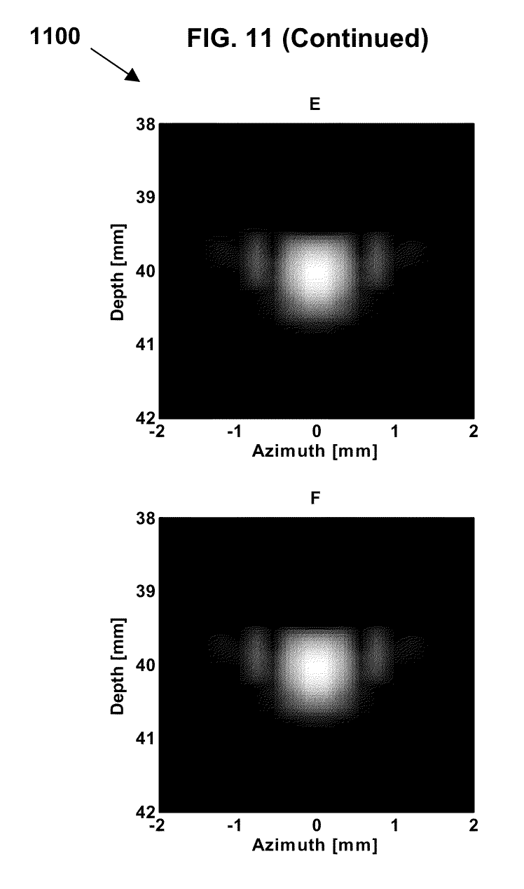

For the simulations, the two outer row arrays transmitted 4 MHz or 8 MHz CW signals and the center-row array received the reflected interference signals. The center-row array was used to obtain echo signals for imaging by a transmission/reception process and then the interference signals were added to the echo signals. The 8 MHz CW signal was regarded as the second harmonic component of the 4 MHz CW signal. In this simulation, a steering angle for CW transmission was fixed assuming the following treatment protocol: The CW beam was focused on a target for a few seconds duration. The −6 dB fractional bandwidths of the imaging and therapy arrays were 50% and 30%, respectively. The bandwidth of the therapy array was lower than that of the imaging array due to the lack of backing of the therapy array. A 4th order Butterworth filter was used to model transfer functions of these transducers in order to carry out more realistic simulation. The band stop attenuation of the notch filter was −37 dB and −31 dB at 4 MHz and 8 MHz, respectively, as shown in FIG. 11, described below.

FIG. 11. depicts a set 1100 of point target simulations (A-F) with imaging signals superimposed with CW interference signals, in accordance with an exemplary embodiment of the present disclosure. All figures were logarithmically compressed with a dynamic range of 40 dB: 2-cycle short pulses, (A) without and (B) with interference; the 13-bit Barker code with 2 cycles per bit, (C) without and (D) with interference; the 13-bit Barker code 5 with 3 cycles per bit, (E) without and (F) with interference. As can be seen, the interference signals mixed with a short pulse signal seriously degraded image quality as presented in FIG. 11B. The Barker code with 3 cycles per bit produced a high SNR image (FIG. 11F) because the interference was greatly suppressed. The 2-cycle-per-bit Barker code generated a high range sidelobe level that primarily appeared around the center scan line (FIG. 11D).

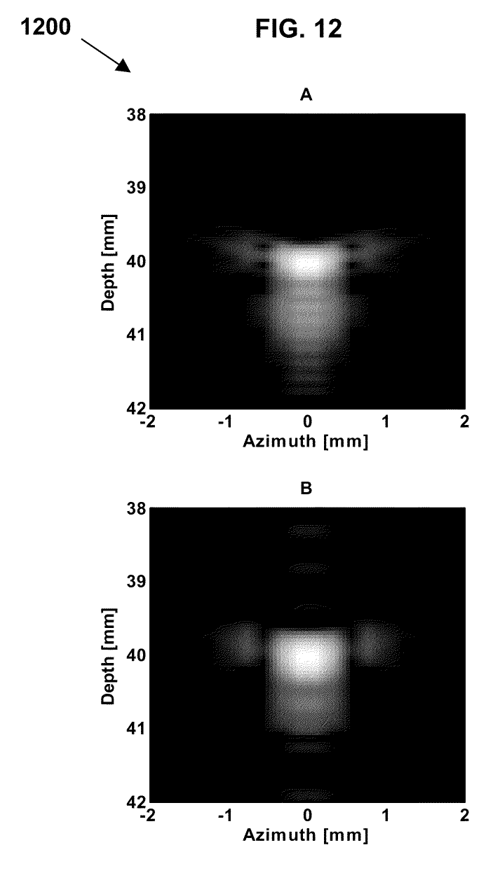

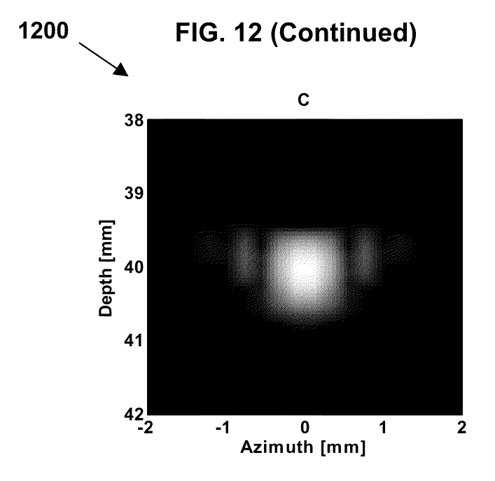

FIG. 12 depicts a set 1200 of simulated point target images of interference-mixed imaging signals after notch filtering, in accordance with an embodiment of the present disclosure. All figures were logarithmically compressed with a dynamic range of 40 dB: (A) 2-cycle short pulses, (B) the 13-bit Barker code with 2 cycles per bit, and (C) the 13-bit Barker code with 3 cycles per bit. FIG. 12 indicates of the performance of the notch filter when the interference was superimposed on the imaging signals. The Barker code with 3 cycles per bit provided a low noise image shown in FIG. 12C which was similar to the image quality without the notch filter (FIG. 11F). The image produced by the short pulse signal shows not only an enhanced range sidelobe level in FIG. 12A due to the notch filter, but also undesired ripples in the axial direction. FIG. 12B illustrates an improved range sidelobe level of the 2-cycle-per-bit Barker code compared to that without notch filtering (FIG. 11D). This point target simulation indicates that a short pulse signal with a notch filter could be used for B-mode imaging, although there are ripples. In reality, it might be difficult to completely remove CW signals as demonstrated by the simulation even using a notch filter. This is because other frequency components around 4 MHz and 8 MHz would be also mixed with the imaging signals. These undesired interference signals increase the range sidelobe level of the envelope signal of the short pulse signal, which was experimentally verified.

### EXAMPLE 4

### Experimental Results

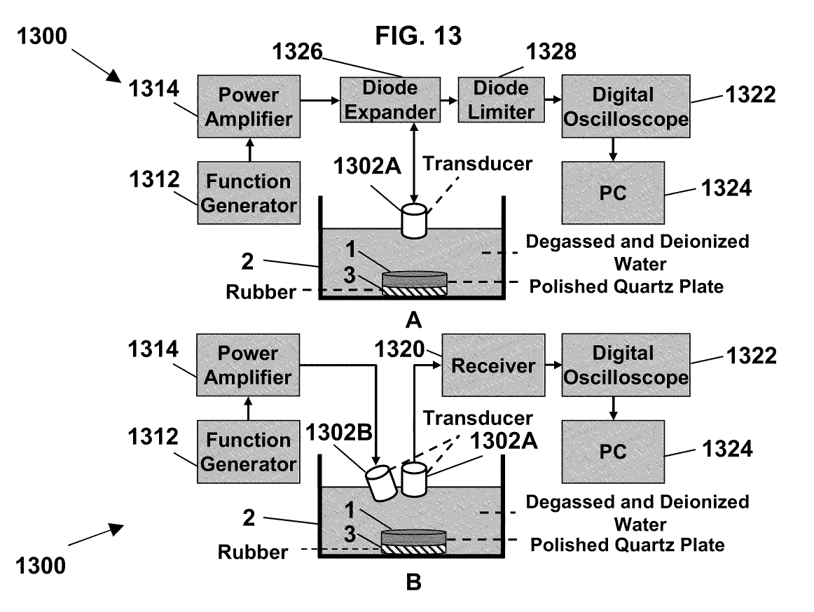

Usually, echo signals contain several types of noises that are different from white noise but can be neglected due to their small amplitude. These noises are associated with the transducer itself, acoustic loads, and electronic components. In the case of HIFU, these interference signals might become significant because of high voltage applied to a HIFU transducer for a rather long duration compared to the case of imaging. Under this situation, the performances of an IMCPA transducer, e.g., transducer 700 of FIG. 7, evaluated with computer simulation described previously, could become degraded. Therefore, the experimental setup in FIG. 13 was constructed to verify whether a coded excitation method with a notch filter could successfully remove the noises as well as reflected therapeutic signals. Because experimental instruments to evaluate the performances of the proposed method under these conditions were not yet available, the inventors utilized commercial single element transducers and equipments for this experiment.

FIG. 13 depicts a diagrammatic view of an experimental setup system 1300 used to collect echo data of (A) imaging signals and (B) CW signals as interference signals, in accordance with exemplary embodiments of the present disclosure. As a target, a polished quartz plate 1 was immersed into a degassed/deionized water tank 2 and a thin rubber layer 3 was placed beneath the quartz plate 1 to minimize reflected signals coming back from the bottom of the water tank. First, as depicted in FIG. 13A, 6 MHz imaging signals were collected by a 5.5 MHz single element transducer 1302A (V308, Olympus, Waltham, Mass.) with a −6 dB fractional bandwidth of 60% (FIG. 13(a)). To acquire 4 MHz CW signals, a 4.5 MHz single element transducer 1302B (IBK5-2, Olympus, Waltham, Mass.) with a −6 dB fractional bandwidth of 50% was used as a transmitter and the 5.5 MHz transducer 1302A as a receiver 20 (FIG. 13(b)). To mimic the second harmonic component of the 4 MHz CW signal, the 8 MHz CW signal was excited by firing a 10 MHz single element transducer (A327R, Olympus, Waltham, Mass.) with a −6 dB fractional bandwidth of 50% and received by the 5.5 MHz transducer. As shown in FIG. 13B, the transmit transducer 1302B was tilted to the receive transducer 1302A. It should be noted that the difference between center frequencies of the commercial transducers (1302A-1302B) and needed frequency components was negligible because the amplitude of all collected data was modified based on Eq. 1 (above).

With continued reference to FIG. 13, a function generator 1312 (33250A, Agilent, Santa Clara, Calif.) was utilized to produce both CW and imaging signals such as 2-cycle short pulses and the 13-bit Barker code with 2 and 3 cycles-per-bit. The transmit signals from the function generator 1312 were sent to a RF power amplifier 1314 (325LA, ENI Co., Santa Clara, Calif.) to boost their amplitude and subsequently used to excite the transducers. A receiver 1320 (5900PR, Panametrics Inc., Waltham, Mass.) 5 and a digital oscilloscope 1322 (LC534, LeCroy, Chestnut Ridge, N.Y.), were used for amplification and recording of echo signals for signal processing with a MATLAB program running (loaded into) on a personal computer (“PC”) 1324. As shown in FIG. 13A, a diode expander 1326 (DEX-3, Matec, Northborough, Mass.) and a diode limiter 1328 (DL-1, Matec, Northborough, Mass.) were used to protect circuits for the system 1300.

The embodiment of FIG. 13, and related testing and simulation, illustrate that techniques of the present disclosure of using coded excitation with/without a notch filter can be applied to not only an array transducer (e.g., transducer 700 of FIG. 7) but also to integrated single-element transducers (with therapy and imaging functionality) and/or separate single-element therapy and imaging transducers (e.g., as depicted in FIG. 13B).

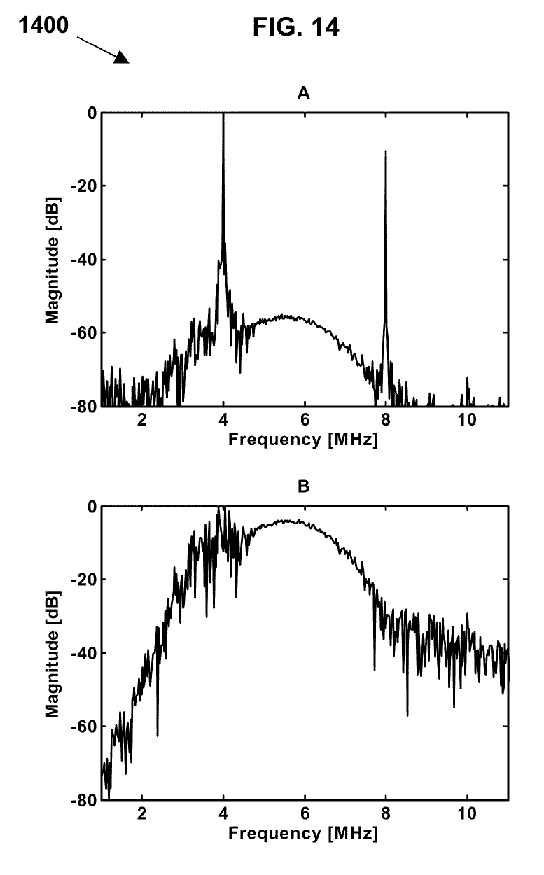

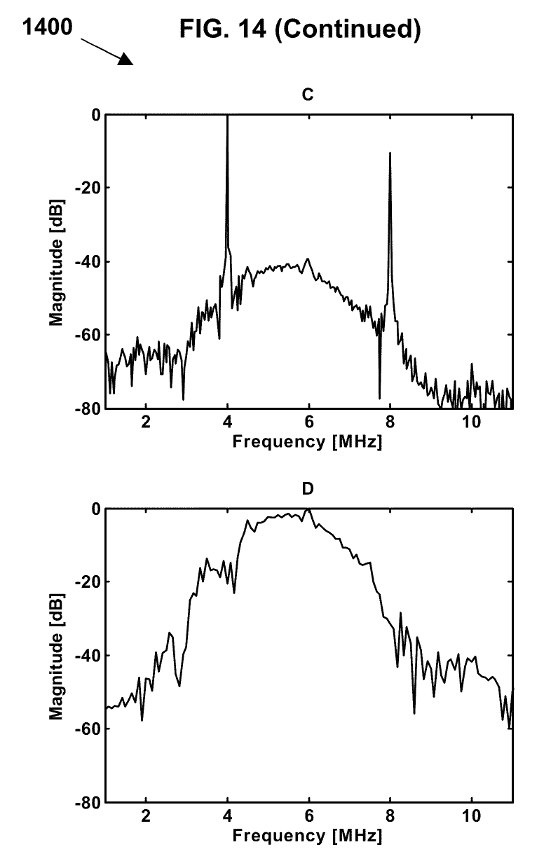

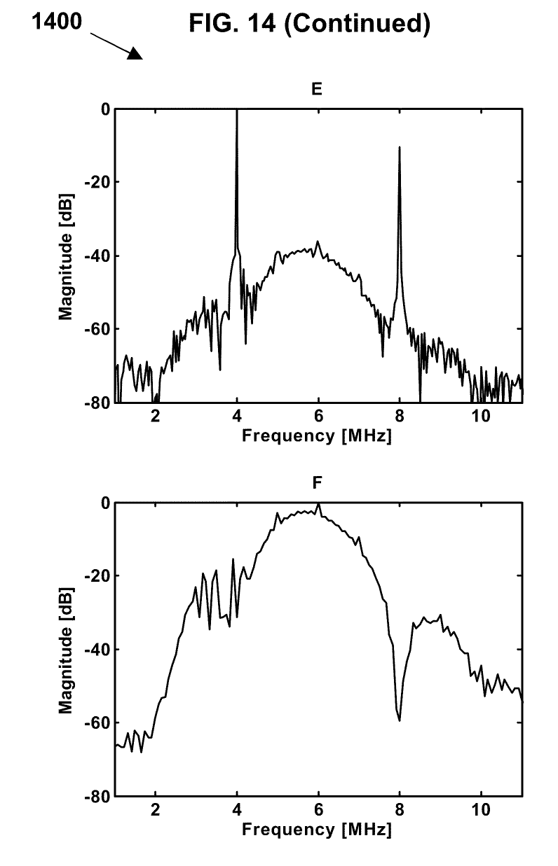

FIG. 14 depicts a set 1400 of graphs (A-F) showing experimental frequency responses of imaging signals with CW interference signals: 2-cycle short pulses, (A) before and (B) after notch filtering; the 13-bit Barker code with 2 cycles per bit, (C) before and (D) after notch filtering; the 13-bit Barker code with 3 cycles per bit, (E) before and (F) after notch filtering. The set 1400 of graphs illustrates the frequency responses of received echo signals (FIGS. 14A, 14C, 14E) and the effect of notch filtering on each spectrum (FIGS. 14B, 14D, 14F). The specifications of the notch filter (e.g., of FIG. 10) described previously were used in this experiment to obtain the results shown in FIG. 14.

With continued reference to FIG. 14, high amplitude interference signals can be discerned at around 4 MHz and 8 MHz frequencies. Although use of a notch filter was shown to dramatically reduce the amplitude of 4 MHz and 8 MHz CW signals mixed with 2-cycle short pulses, spurious signals around 4 MHz and 8 MHz frequencies still remained as shown in FIG. 14B. It is clear from FIG. 14D and FIG. 14F that the 13-bit Barker codes with 2 and 3 cycles per bit have high robustness in suppressing the noises, thus providing the SNR improvement after pulse compression.

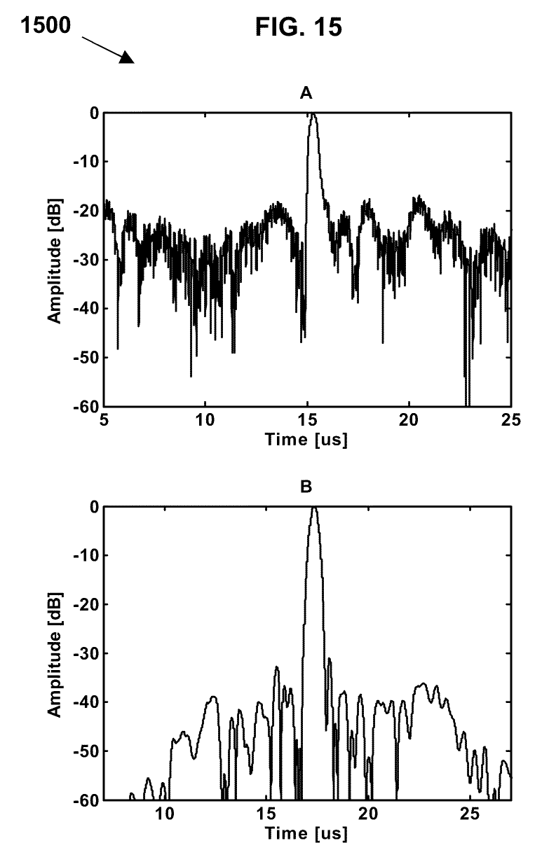

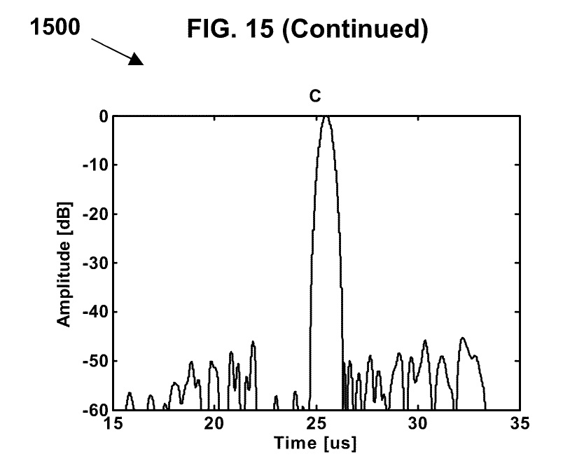

FIG. 15 depicts a set 1500 of graphs (A-C) showing experimental envelope signals: (A) 2-cycle short pulses, (B) the 13-bit Barker code with 2 cycles per bit after pulse compression, and (C) the 13-bit Barker code with 3 cycles per bit after pulse compression. These undesired interference signals were not readily removed by a notch filter. As a result, the envelope of 2-cycle short pulses in FIG. 15A had a range sidelobe level of ˜18 dB due to the remaining interference. The 2- and 3-cycle-per-bit Barker code offered a range sidelobe level of −40 dB and −48 dB after pulse compression as shown in FIG. 15B and FIG. 15C, respectively. Note that the 13-bit Barker code with 3 cycles per bit can also provide the best range sidelobe level without the notch filtering, which was about −47 dB.

To compare the axial resolution, −6 dB and −20 dB axial beamwidths were measured and the results were summarized in Table I. The 2-cycle-per-bit Barker code had a −6 dB axial beamwidth of 0.39 mm which was 0.02 mm wider than that of the 2-cycle short pulses, but 0.13 mm narrower than that of the 3-cycle-per-bit Barker code. The −20 dB axial beamwidth of the 2-cycle-per-bit Barker code was 0.4 mm narrower than that of the 3-cycle-per-5 bit Barker code. However, the −20 dB axial beamwidth of 2-cycle short pulses could not be measured because its range sidelobe level was around −18 dB, so that it could not be used for imaging in spite of notch filtering.

TABLE 1
Experimental −6 dB axial beamwidths, −20 dB axial beamwidths,
and range sidelobe levels of three different imaging signals

| | | 13-bit | 13-bit |
|---|---|---|---|
| | | Barker code | Barker code |
| | Pulse | (2 cycles | (3 cycles |
| | (2 cycles) | per bit) | per bit) |
| −6 dB axial beamwidth (mm) | 0.37 | 0.39 | 0.52 |
| −20 dB axial beamwidth (mm) | — | 0.67 | 0.91 |
| Range sidelobe level (dB) | −18 | −40 | −48 |

Thus, coded excitation (with or without a notch filter) techniques and transducer configurations (e.g., integrated multi-functional confocal phased array) of the present disclosure can be advantageously applied to desired targets. Exemplary embodiments of the present disclosure are beneficially applicable to the treatment and imaging of any target such as breast, liver, prostate, and so on. For example, 1 MHz˜2 MHz frequencies can be used for HIFU treatment of human liver and its focal depth is about 15 cm. In the case of prostate tissue, 3 MHz˜4 MHz frequencies can be used for HIFU and its focal depth is 4 cm˜5 cm. By combining two therapy arrays and one imaging array, the fabrication complexity of an IMCPA may be decreased, and each array may maintain its own optimal performance. The confocal structure of an IMCPA in the elevational direction may improve the detection capability. Simulation and experimental results obtained by the inventors verify that coded excitation and/or a notch filter may be able to improve the range sidelobe level of the B-mode image during therapy.

Accordingly, relative to other techniques, embodiments of the present disclosure can provide for various advantages including, but not limited to, one or more of the following:
a. true real-time simultaneous treatment and monitoring is possible; the performance of treatment and imaging can be preserved by using separate arrays;
b. the amplitude of reflected therapeutic fundamental or harmonic signals can be reduced because of an angle difference between therapeutic array and imaging array due to its confocal structure in the elevational direction.
c. array configuration makes it possible to carry out dynamic focusing and steering by electrical delay control;
d. the fabrication complexity can be decreased greatly by integrating two different kinds of transducers which are already made;
e. the coded excitation technique with/without a notch filter for imaging can minimize the interference of reflected therapeutic signal;
f. the different piezoelectric materials and stack configurations can be employed along with their functionalities, i.e. therapy or imaging in order to maximize their performances
g. the thermal effect of HIFU can be maintained by using PW, CW, or coded signal like chirps;
h. the grating lobe effect can be reduced by using chirp signal for treatment;
i. the cavitational effect of ultrasound can be minimized by using chirp signal for treatment;
j. techniques of the present disclosure can be applied to real-time simultaneous therapy and diagnosis based on coded harmonic imaging

While certain embodiments and/or aspects have been described herein, it will be understood by one skilled in the art that other embodiments may be included within the scope of the present disclosure. For example, while various implementation parameters are described herein, embodiments of the present disclosure can be used for various other situations/applications, such as different power levels of therapeutic signals, different duty factors of therapy or imaging signals, and different pulse repetition frequencies (“PRF”). Further, while Barker codes have been described for exemplary embodiments, techniques of the present disclosure are not limited to such and other pulse compression techniques can be used within the scope of the present disclosure. Suitable examples include, but are not limited to, general binary-phase-coded pulse compression, linear recursive sequences (or shift register codes), Golay or complimentary codes, quadriphase codes, polyphase codes, so-called “combined,” “concatenated,” or “compound” Barker codes and the like, as well as frequency modulation techniques including chirps (linear or non-linear) and the like.

Additionally, the piezoelectric materials and composite(s) utilized for embodiments described herein are merely representative and others may be used. The coded excitation (with or without a notch filter) can be used for any types of integrated multi-functional confocal transducers: not only array transducers such as linear, phased, convex, and concave arrays but also single element transducers. One dimensional and two dimensional transducers can also be employed in this configuration. Accordingly, the embodiments described herein, and as claimed in the attached claims, are to be considered in all respects as illustrative of the present disclosure and not restrictive.

What is claimed is:

1. An ultrasound system comprising:
an acoustic therapy array;
an acoustic imaging array; and
a controller system configured and arranged to (i) control the acoustic therapy array to produce an ultrasonic output for therapeutic ultrasound treatment of a targeted portion of tissue, and (ii) control the acoustic imaging array to produce a pulse-compressed ultrasonic output for imaging the targeted portion of tissue during treatment.

2. The system of claim 1, wherein the imaging array comprises a linear, phased, convex, and concave array of PZT imaging transducers.

3. The system of claim 1, wherein the imaging array comprises one dimensional or two dimensional arrays.

4. The system of claim 1, wherein the therapy array comprises one or more linear, phased, convex, and concave arrays of therapy transducers.

5. The system of claim 1, wherein the therapy array comprises one dimensional or two dimensional arrays.

6. The system of claim 2, wherein each imaging transducer comprises a PZT layer adjacent to a matching layer and a backing layer.

7. The system of claim 2, wherein each therapy transducer comprises a PZT layer with a matching layer.

8. The system of claim 4, comprising two therapy arrays configured on respective sides of the imaging array.

9. The system of claim 5, comprising four or more therapy arrays configured with two or more arrays on respective sides of the imaging array.

10. The system of claim 1, wherein the controller system is configured and arranged to control the acoustic imaging array to produce a pulse-compressed ultrasonic output including a Barker code, a chirp signal, or a Golay code.

11. The system of claim 1, wherein the controller system comprises a therapy circuit configured and arranged to control the output of the therapy array.

12. The system of claim 1, wherein the controller system comprises an imaging circuit configured and arranged to control the output of the imaging array, and wherein the imaging circuit includes a pulse compressor for compressing pulses on transmit.

13. The system of claim 12, further comprising a means for display configured and arranged to receive signals from the display circuit of the controller system and display an image of the targeted portion of tissue.

14. A method of simultaneous ultrasonic imaging and treatment of targeted tissue, the method comprising:
controlling an acoustic therapy array to produce an ultrasonic output for therapeutic ultrasound treatment of a targeted portion of tissue;
controlling an acoustic imaging array to produce a pulse-compressed ultrasonic output for imaging the targeted portion of tissue during treatment;
receiving ultrasonic energy reflected from the targeted tissue; and
imaging and displaying an ultrasound image of the targeted tissue.

15. The method of claim 14, wherein controlling an acoustic image array to produce a pulse-compressed ultrasonic output comprises using a binary phase-coded pulse.

16. The method of claim 15, wherein using a binary phase-coded pulse comprises using a Barker code.

17. The method of claim 16, wherein the Barker code is of 2, 3, 4, 5, 7, 11, or 13.

18. The method of claim 17, wherein the Barker code is of length 13.

19. The method of claim 14, wherein controlling an acoustic image array to produce a pulse-compressed ultrasonic output comprises using a chirp signal.

20. The method of claim 15, wherein using a binary phase-coded pulse comprises using a Golay code.

21. The method of claim 15, wherein using a binary phase-coded pulse comprises using a linear recursive sequence.

22. The method of claim 15, wherein using a binary phase-coded pulse comprises using a quadriphase code.

23. The method of claim 14, wherein controlling an acoustic therapy array to produce an ultrasonic output for therapeutic ultrasound treatment comprises controlling the acoustic therapy array to produce a chirped acoustic output pulse.

24. The method of claim 14, wherein controlling an acoustic therapy array to produce an ultrasonic output for therapeutic ultrasound treatment comprises controlling the acoustic therapy array to produce a continuous wave output for a desired time.

25. The method of claim 14, wherein controlling an acoustic therapy array to produce an ultrasonic output for therapeutic ultrasound treatment comprises controlling the acoustic therapy array to produce an acoustic output from two or more therapy arrays.

26. An ultrasonic integrated multi-functional confocal phased array (IMCPA) comprising:
an imaging array including a phased array having at least one row of acoustic imaging transducers configured and arranged to send acoustic energy to a focal spot; and
two or more therapy arrays, wherein each therapy array includes a phased array having at least one row of acoustic therapy transducers configured and arranged to send acoustic energy to the focal spot.

27. The array of claim 26, wherein the imaging transducers comprise a 1-3 piezocomposite.

28. The array of claim 27, wherein the piezocomposite comprises PZT-5H with epoxy.

29. The array of claim 26, wherein the imaging array is configured and arranged for operation at about 4 MHz to about 8 MHz.

30. The array of claim 29, wherein the imaging array is configured and arranged for operation at about 6 MHz.

31. The array of claim 26, wherein each imaging transducer comprises a PZT layer adjacent to a matching layer and a backing layer.

32. The array of claim 26, wherein the two or more therapy arrays are configured on respective sides of the imaging array.

33. The array of claim 26, wherein the two or more therapy arrays comprise a 1-3 piezocomposite.

34. The array of claim 33, wherein the piezocomposite comprises PZT4 or PZT8 with high thermal resistance epoxy.

35. The array of claim 26, wherein the two or more therapy arrays are configured and arranged to operate at about 1 MHz to about 5 MHz.

36. The array of claim 35, wherein the wherein the two or more therapy arrays are configured and arranged to operate at about 4 MHz.

37. The array of claim 36, wherein the array is configured and arranged to produce a therapy intensity of about 2000 W/cm² at the focal spot.

38. A method of simultaneous ultrasonic imaging and treatment of targeted tissue, the method comprising:
controlling one or more acoustic transducers to produce an ultrasonic output for therapeutic ultrasound treatment of a targeted portion of tissue;
controlling an acoustic imaging transducer to produce a coded ultrasonic output for imaging the targeted portion of tissue during treatment;
with the imaging transducer, receiving ultrasonic energy reflected from the targeted tissue; and
imaging and displaying an ultrasound image of the targeted tissue.

39. The method of claim 38, further comprising using a notch filter to filter the ultrasonic energy received by the imaging transducer.
